(19) United States
(12) Patent Application Publication
(10) Pub. No.: US 2005/0239813 A1
(43) Pub. Date: Oct. 27, 2005

(54) Pharmaceutical compositions comprising fk506 derivatives and their use for the treatment of allergic diseases

(75) Inventor: Ryuji Ueno, Montgomery, MD (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Sucampo Pharmaceuticals Inc., Bethesda, MD (US)

(21) Appl. No.: 10/523,842

(22) PCT Filed: Aug. 8, 2003
(86) PCT No.: PCT/JP03/10105

§ 371 Date: Jun. 13, 2005

(30) Foreign Application Priority Data
Aug. 9, 2002 (US) 60402061

(51) Int. Cl. A61K 31/4745

(52) U.S. Cl. 514/291

(57) ABSTRACT

The invention provides ophthalmic compositions and methods treating the symptoms of ocular allergies. The principle active ingredient in these compositions and methods is macrolide compound, such as tacrolimus, ascomycin and rapamycin and their derivatives. Optimal concentrations and dosing regimens are provided.

### TECHNICAL FIELD OF THE INVENTION

The present invention relates to a method for treating ocular allergies.

### BACKGROUND ART

The incidence and prevalence of allergic conjunctivitis has increased dramatically over the past 40 years, today affecting up to 20% of the US population. The condition can be seasonal if due to pollens from trees, grasses or weeds, or perennial, if the antigen is abundant throughout the year such as animal dander, dust or mold; of the two, seasonal allergic conjunctivitis is more common. Ophthalmologists believe that 80%-90% of all allergic conjunctivitis cases are seasonal, while the remaining 10%-20% are perennial in nature.

Ocular allergies, like allergic conjunctivitis are currently being served with products within the following categories: antihistamines, mast cell stabilizers, nonsteroidal anti-inflammatory drugs (“NSAIDS”) and corticosteroids. Despite the availability of so many products, none of them are entirely satisfactory and there still exists a need for products that are effective and work using different mechanisms of action.

Historically, there has been some interest in the development of macrolide immunosuppressive compounds in to treat allergic conjunctivitis, but as yet there is no product on the market. Of these macrolide compounds, notable is tacrolimus, aka FK506, originated by Fujisawa Pharmaceutical Co., Ltd. in Japan. See U.S. Pat. No. 5,514,686. Heretofore, however, there has never been defined an optimal dosing regimen for treating human patients.

### DISCLOSURE OF THE INVENTION

It is, therefore, an object of the invention to provide useful, improved compositions and methods for treating ocular allergy symptoms with macrolide immunosuppressive compounds.

An another object of the invention to provide a commercial package comprising the composition of the present invention and a written matter associated therewith, the written matter stating the composition can or should be used for ocular allergies, especially allergic conjunctivitis.

According to this and other objects of the invention, a method of treating a human patient suffering from ocular allergy symptoms is provided. According to one embodiment, this method entails administering to the patient an ophthalmic composition containing from about 0.01% to about 0.1% of a macrolide compound. In other embodiments, the method involves administering to the patient an ophthalmic composition containing from about 0.03% to about 0.06% of a macrolide compound, but preferably about 0.03%.

Preferred compositions are formulated as eye drops, which optionally contain polyvinyl alcohol, or ointments. In general, these compositions will be administered to the eye from about one to about four times per day.

Preferred macrolide compound is a tricyclo compound having the following formula (I) or a pharmaceutically acceptable salt thereof: 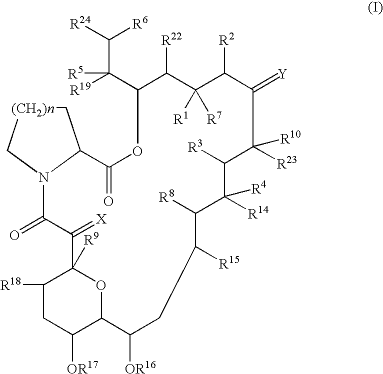
wherein adjacent pairs of R¹ and R², R³ and R⁴, and R⁵ and R⁶ each independently: (a) consist of two adjacent hydrogen atoms, wherein R² is optionally alkyl, or (b) form another bond optionally between carbon atoms binding with the members of said pairs; R⁷ is hydrogen atom, hydroxy, alkyloxy or protected hydroxy, or may form oxo with R¹; R⁸ and R⁹ each independently show hydrogen atom or hydroxy; R¹⁰ is hydrogen atom, alkyl, alkyl substituted by one or more hydroxy, alkenyl, alkenyl substituted by one or more hydroxy or alkyl substituted by oxo; X is oxo, (hydrogen atom, hydroxy), (hydrogen atom, hydrogen atom), or a group of the formula —CH₂O—; Y is oxo, (hydrogen atom, hydroxy), (hydrogen atom, hydrogen atom), or a group of the formula N—NR¹¹R¹² or N—OR¹³; R¹¹ and R¹² each independently show hydrogen atom, alkyl, aryl or tosyl; R¹³, R¹⁴, R¹⁵, R¹⁶, R¹⁷, R¹⁸, R¹⁹, R²² and R²³ each independently show hydrogen atom or alkyl; R²⁴ is an optionally substituted ring that may contain one or more hetero atom(s); and n is 1 or 2. Tacrolimus is most preferred.

### BRIEF DESCRIPTION OF THE DRAWING

FIG. 1 shows the ability of macrolide-containing eye drops to suppress ocular itching in humans in response to challenge with common allergens.

### DETAILED DESCRIPTION OF THE INVENTION

The present inventor has surprisingly discovered that certain macrolide compounds can be used in specific concentration ranges to treat the ocular symptoms of allergy. In particular, macrolide compounds like FK506 (tacrolimus), ascomycin, rapamycin and their derivatives, can be used in concentrations ranging from about 0.01% to about 0.1% in ophthalmic compositions to treat ocular allergy symptoms and, in particular, allergic conjunctivitis.

Macrolide Compounds of the Invention

A specific example of a macrolide compound usable in the invention is a tricyclo compound as shown by the following general formula (I) or a pharmaceutically acceptable salt thereof. 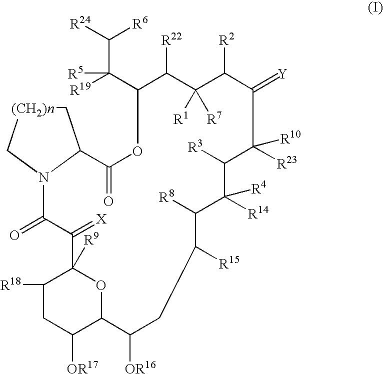
wherein adjacent pairs of R¹ and R², R³ and R⁴, and R⁵ and R⁶ each independently
a) consist of two adjacent hydrogen atoms, wherein R² is optionally alkyl, or
b) form another bond optionally between carbon atoms binding with the members of said pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkyl substituted by one or more hydroxy, alkenyl, alkenyl substituted by one or more hydroxy or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s) and;
n is 1 or 2.

In addition to the meaning noted above, Y, R¹⁰ and R²³ may show, together with the carbon atom they bind with, a saturated or unsaturated 5 or 6-membered heterocyclic group containing nitrogen atom, sulfur atom and/or oxygen atom, the heterocyclic group being optionally substituted by one or more group(s) selected from alkyl, hydroxy, alkyloxy, benzyl, a group of the formula —CH₂Se(C₆H₅), and alkyl substituted by one or more hydroxy, or its pharmaceutically acceptable salt.

In the general formula (I), preferably R²⁴ is, for example, cyclo(C₅-C₇)alkyl optionally having suitable substituent, such as the following.
(a) 3,4-dioxocyclohexyl
(b) 3-R²⁰-4-R²¹-cyclohexyl,
   wherein R²⁰ is hydroxy, alkyloxy or —OCH₂OCH₂CH₂OCH₃, and R²¹ is hydroxy, —OCN, alkyloxy, heteroaryloxy optionally having suitable substituent, —OCH₂OCH₂CH₂OCH₃, protected hydroxy, chloro, bromo, iodo, aminooxalyloxy, azide, p-tolyloxythiocarbonyloxy, or R²⁵R²⁶CHCOO— (wherein R²⁵ is hydroxy optionally protected where desired or protected amino, and R²⁶ is hydrogen atom or methyl, or R²⁰ and R²¹ in combination form an oxygen atom of epoxide ring or
(c) cyclopentyl wherein cyclopentyl is substituted by methoxymethyl, optionally protected hydroxymethyl where desired, acyloxymethyl (wherein acyl moiety is optionally quaternized dimethylamino or optionally esterified carboxy), one or more optionally protected amino and/or hydroxy, or aminooxalyloxymethyl. Preferable examples include 2-formyl-cyclopentyl.

The definition of each symbol used in the formula (I), specific examples thereof and preferable embodiments thereof will be explained in detail in the following.

“Lower” generally means a group having from about 1 to about 6 carbon atoms unless otherwise indicated.

Preferable examples of the alkyl moiety of “alkyl” and “alkyloxy” include linear or branched aliphatic hydrocarbon residue, such as lower alkyl (e.g., methyl, ethyl, propyl, isopropyl, butyl, isobutyl, pentyl, neopentyl, hexyl and the like).

Preferable examples of “alkenyl” include linear or branched aliphatic hydrocarbon residue having one double bond, such as lower alkenyl (e.g., vinyl, propenyl (e.g., allyl and the like), butenyl, methylpropenyl, pentenyl, hexenyl and the like).

Preferable examples of “aryl” include phenyl, tolyl, xylyl, cumenyl, mesityl, naphthyl and the like.

Preferable examples of the protective group for “protected hydroxy” and “protected amino” include 1-(lower alkylthio)(lower)alkyl such as lower alkylthiomethyl (e.g., methylthiomethyl, ethylthiomethyl, propylthiomethyl, isopropylthiomethyl, butylthiomethyl, isobutylthiomethyl, hexylthiomethyl and the like), with more preference given to C₁-C₄ alkylthiomethyl and most preference given to methylthiomethyl; tri-substituted silyl such as tri(lower)alkylsilyl (e.g., trimethylsilyl, triethylsilyl, tributylsilyl, tert-butyl dimethylsilyl, tri-tert-butylsilyl and the like), and lower alkyldiarylsilyl (e.g., methyldiphenylsilyl, ethyldiphenylsilyl, propyldiphenylsilyl tert-butyldiphenylsilyl and the like), with more preference given to tri(C₁-C₄)alkylsilyl and C₁-C₄ alkyldiphenylsilyl, and most prefererence given to tert-butyl-dimethylsilyl and tert-butyldiphenylsilyl; acyl such as aliphatic acyl, aromatic acyl and aliphatic acyl substituted by aromatic group, which are derived from carboxylic acid, sulfonic acid and carbamic acid; and the like.

The aliphatic acyl is exemplified by lower alkanoyl optionally having one or more suitable substituent(s) (e.g., carboxy) such as formyl, acetyl, propionyl, butyryl, isobutyryl, valeryl, isovaleryl, pivaloyl, hexanoyl, carboxyacetyl, carboxypropionyl, carboxybutyryl, carboxyhexanoyl and the like; cyclo(lower)alkyloxy(lower)alkanoyl optionally having one or more suitable substituent(s) (e.g., lower alkyl) such as cyclopropyloxyacetyl, cyclobutyloxypropionyl, cycloheptyloxybutyryl, mentyloxyacetyl, mentyloxypropionyl, mentyloxybutyryl, mentyloxypentanoyl, mentyloxyhexanoyl and the like; camphorsulfonyl; lower alkylcarbamoyl having one or more suitable substituent(s) such as carboxy or protected carboxy and the like, such as carboxy(lower)alkylcarbamoyl (e.g., carboxymethylcarbamoyl carboxyethylcarbamoyl, carboxypropylcarbamoyl, carboxybutylcarbamoyl, carboxypentylcarbamoyl, carboxyhexylcarbamoyl) and tri(lower)alkylsilyl(lower)alkyloxycarbonyl(lower)alkylcarbamoyl (e.g., trimethylsilylmethoxycarbonylethylcarbamoyl, trimethylsilylethoxycarbonylpropylcarbamoyl, triethylsilylethoxycarbonylpropylcarbamoyl, tert-butyl dimethylsilylethoxycarbonylpropylcarbamoyl, trimethylsilylpropoxycarbonylbutylcarbamoyl.

Aromatic acyl is exemplified by aroyl optionally having one or more suitable substituent(s) (e.g., nitro), such as benzoyl, toluoyl, xyloyl, naphthoyl, nitrobenzoyl, dinitrobenzoyl, nitronaphthoyl and the like and arenesulfonyl optionally having one or more suitable substituent(s) (e.g., halogen), such as benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl and the like.

The aliphatic acyl substituted by aromatic group may be, for example, ar(lower)alkanoyl optionally having one or more suitable substituent(s) (e.g., lower alkyloxy or trihalo(lower)alkyl and the like), wherein specific examples are phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl and the like.

Of the above-mentiond acyl, more preferable acyl includes C₁-C₄ alkanoyl optionally having carboxy, cyclo(C₅-C₆)alkyloxy(C₁-C₄)alkanoyl having two (C₁-C₄)alkyl in the cycloalkyl moiety, camphorsulfonyl, carboxy(C₁-C₄)alkylcarbamoyl, tri(C₁-C₄)alkylsilyl(C₁-C₄)alkyloxycarbonyl(C₁-C₄)alkylcarbamoyl, benzoyl optionally having one or two nitro groups, and benzenesulfonyl having halogen, phenyl(C₁-C₄)alkanoyl having C₁-C₄alkyloxy and trihalo(C₁-C₄)alkyl. Of these, most preferred are acetyl, carboxypropionyl, mentyloxyacetyl, camphorsulfonyl, benzoyl, nitrobenzoyl, dinitrobenzoyl, iodobenzenesulfonyl, 2-trifluoromethyl-2-methoxy-2-phenylacetyl and the like.

Preferable examples of the “heterocyclic group consisting of saturated or unsaturated 5 or 6-membered ring having nitrogen atom, sulfur atom and/or oxygen atom” are pyrolyl, tetrahydrofuryl and the like.

The “heteroaryl optionally having suitable substituent moiety” of the “heteroaryloxy optionally having suitable substituent” is that exemplified for R¹ of the compound of the formula I of EP-A-532,088, with preference given to 1-hydroxyethylindol-5-yl. The disclosure is incorporated hereinto by reference.

The tricyclo compound (I) used in the present invention is described in the publications EP-A-184162, EP-A-323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/5059 and the like. The disclosures of these publications are incorporated herein by reference.

In particular, the compounds called FR900506 (FK506), FR900520 (Ascomycin), FR900523 and FR900525 are produced by the genus Streptomyces, such as Streptomyces tsukubaensis, No. 9993 (depository: National Institute of Advanced Industrial Science and Technology, International Patent Organism Depositary, Central 6, 1-1, Higashi 1-chome, Tsukuba-shi, Ibaraki-ken, Japan (formerly Fermentation Research Institute, Agency of Industrial Science and Technology, the Ministry of International Trade and Industry), date of deposit: Oct. 5, 1984, deposit number FERM BP-927) or Streptomyces hygroscopicus subsp. Yakushimaensis, No. 7238 (depository National Institute of Advanced Industrial Science and Technology, International Patent Organism Depositary, Central 6, 1-1, Higashi 1-chome, Tsukuba-shi, Ibaraki-ken, Japan (formerly Fermentation Research Institute, Agency of Industrial Science and Technology, the Ministry of International Trade and Industry), date of deposit Jan. 12, 1985, deposit number: FERM BP-928 (EP-A-0184162)), and the compound of the following formula, FK506 (generic name: Tacrolimus) is a representative compound.

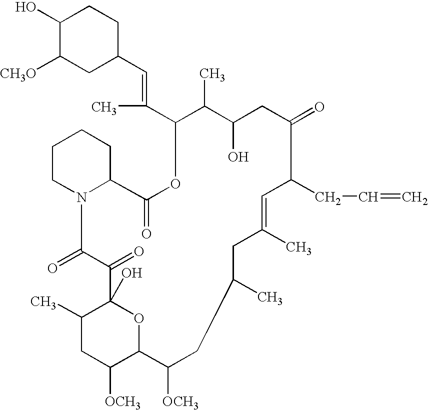

### Chemical name: 17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetraone

Of the tricyclo compounds (1), more preferred is a compound wherein adjacent pairs of R³ and R⁴, and R⁵ and R⁶ each independently form another bond optionally between carbon atoms binding with the members of said pairs;
R⁸ and R²³ each independently show hydrogen atom;
R⁹ is hydroxy;
R¹⁰ is methyl, ethyl, propyl or allyl;
X is (hydrogen atom, hydrogen atom) or oxo;
Y is oxo;
R¹⁴, R¹⁵, R¹⁶, R⁷, R⁸, R¹⁹ and R²² each independently show methyl;
R²⁴ is 3-R²⁰-4-R²¹-cyclohexyl,
wherein R²⁰ is hydroxy, alkyloxy or —OCH₂OCH₂CH₂OCH₃, and R²¹ is hydroxy, —OCN, alkyloxy, heteroaryloxy optionally having suitable substituent, —OCH₂OCH₂CH₂OCH₃, protected hydroxy, chloro, bromo, iodo, aminooxalyloxy, azide, p-tolyloxythiocarbonyloxy or R²⁵R²⁶CHCOO— (wherein R²⁵ is optionally protected hydroxy as desired, or protected amino, and R²⁶ is hydrogen atom or methyl), or R²⁰ and R²¹ in combination form an oxygen atom of epoxide ring; and
n is 1 or 2.

Particularly preferable tricyclo macrolide compounds (I) include, besides FK506, Ascomycin derivatives such as halogenated derivative of 33-epi-chloro-33-desoxy Ascomycin described in Example 66a of EP-A-427,680 and the like.

Other preferable macrolide compounds include Rapamycin described in MERCK INDEX, 12 edition, No. 8288 and derivatives thereof. Preferable examples thereof include O-substituted derivative described at page 1 of WO95/16691, formula A, wherein the 40^{th} hydroxy is —OR₁ (wherein R₁ is hydroxyalkyl, hydroalkyloxyalkyl, acylaminoalkyl and aminoalkyl), such as 40-O-(2-hydroxy)ethyl Rapamycin, 40-O-(3-hydroxy)propyl Rapamycin, 40-O-[2-(2-hydroxy)ethoxy]ethyl Rapamycin and 40-O-(2-acetaminoethyl)-Rapamycin. These O-substituted derivatives can be produced by reacting, under appropriate conditions, Rapamycin (or dihydro or deoxo Rapamycin) and an organic radical bound with leaving group (e.g., RX wherein R is an organic radical desirable as O-substituent, such as alkyl, allyl and benzyl moiety, and X is a leaving group such as CCl₃C(NH)O and CF₃SO₃)). The conditions are: when X is CCl₃C(NH)O, acidic or neutral conditions, such as in the presence of trifluoromethanesulfonic acid, camphorsulfonic acid, p-toluenesulfonic acid or their corresponding pyridinium or substituted pyridinium salt, and when X is CF₃SO₃, in the presence of a base such as pyridine, substituted pyridine, diisopropylethylamine and pentamethylpiperidine. The most preferable Rapamycin derivative is 40-O-(2-hydroxy)ethyl Rapamycin as disclosed in WO94/09010, which is hereby incorporated into the specification by reference.

The pharmaceutically acceptable salt of tricyclo compound (I), Rapamycin and derivatives thereof are nontoxic and pharmaceutically acceptable conventional salts, which are exemplified by salts with inorganic or organic base such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

The macrolide compound of the invention comprises one or more pairs of stereoisomers, such as optical isomers and geometric isomers, which may be included due to conformers or asymmetric carbon atoms and double bonds. Such conformers and isomers are also encompassed in the present invention. In addition, macrolide compounds can form solvates, which also are encompassed by the present invention. Preferable solvates include hydrates and ethanolates.

The instant macrolide compounds and their pharmaceutically acceptable salts are nontoxic. Pharmaceutically acceptable conventional salts may have an inorganic or organic base, such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

As used herein, unless otherwise specifically noted, the term “macrolide” or reference to a particular macrolide is meant to include all pharmaceutically acceptable salts thereof.

Ophthalmic Compositions

While the present macrolide compounds may be administered any number of ways, the most convenient forms are contemplated to be eye drops and ointments, which may be prepared according to conventional methods. The optimal concentration of the macrolide compounds is in the range of about 0.01% to about 0.1% (more strictly, 0.01% to 0.1%), but more preferably is about 0.03% to about 0.06% (more strictly, 0.03% to 0.06%), with 0.03% being most preferred.

Eye drops, for instance, may be prepared by dissolving the active ingredient in a sterile aqueous solution such as physiological saline, buffering solution, etc., or by providing a powdered composition that is dissolved before use. Eye drops such as the ones as described in EP-A-0406791 (which is incorporated by reference in its entirety) are preferred. Conventional eye drop additives can be used. Such additives include isotonizing agents (e.g., sodium chloride, etc.), buffer agents (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, etc.; e.g., polyvinyl alcohol, methylcellulose, glycerine, etc.).

Especially, polyvinyl alcohol as additive is preferably used in the eye drop of the present invention.

Ophthalmic ointments may be prepared by mixing the active ingredient with a base according to conventional methods. Examples ointment bases include, but are not limited to, petrolatum, selen 50, Plastibase and macrogol. In order to increase the hydrophilicity, a surface-active agent, like a detergent or other emulsifier, can be added. The same additives used in the eye drops, such as the preservatives, etc. can also be used in an ointment.

The present formulation can further include other pharmacological active ingredients as far as they do not contradict the purpose of the present invention. For instance, the formulation can include a single or multiple macrolide compounds, and may also include one or more antimicrobial agents as active ingredients for the purpose of treating or preventing bacterial infections. In a combination of plural active ingredients, their respective contents may be suitably increased or decreased in consideration of their effects and safety.

The present agent can be formulated as a sterile unit dose type containing no preservatives.

Methods of Treatment

The term “treatment” used herein includes any means of control such as prevention, care, relief of the condition, attenuation of the condition and arrest of progression.

The patient being treated will generally have a history of ocular allergy symptoms. Most pronounced among those symptoms are redness and itching. The patient may be suffering from allergic conjunctivitis.

The present macrolide-containing compositions, described above, generally are topically administered to the eyes and/or the surrounding skin, such as the eyelids. The amount and frequency of administration can vary according to sex, age and weight of a human, symptoms to be treated, desirable therapeutic effects, administration routes and period for treatment. However, the inventor has found the optimal concentration of macrolide compound in the ophthalmic composition (eye drop, eye ointment) for treating ocular allergies to be in the range of about 0.01% to about 0.06%. Concentrations of up to about 0.1% may be used, but generally those are best formulated as an ointment. When considering all factors, concentrations of 0.03% appear to be best suited for treatment. Preferably, the macrolide compounds is formulated as an eye drop and may be administered several times a day per eye, preferably one to six times, more preferably one to four times, several drops per time, preferably one to four drops.

The present invention will be described in more detail with reference to the following examples, which are not intended to limit the present invention.

### EXAMPLES

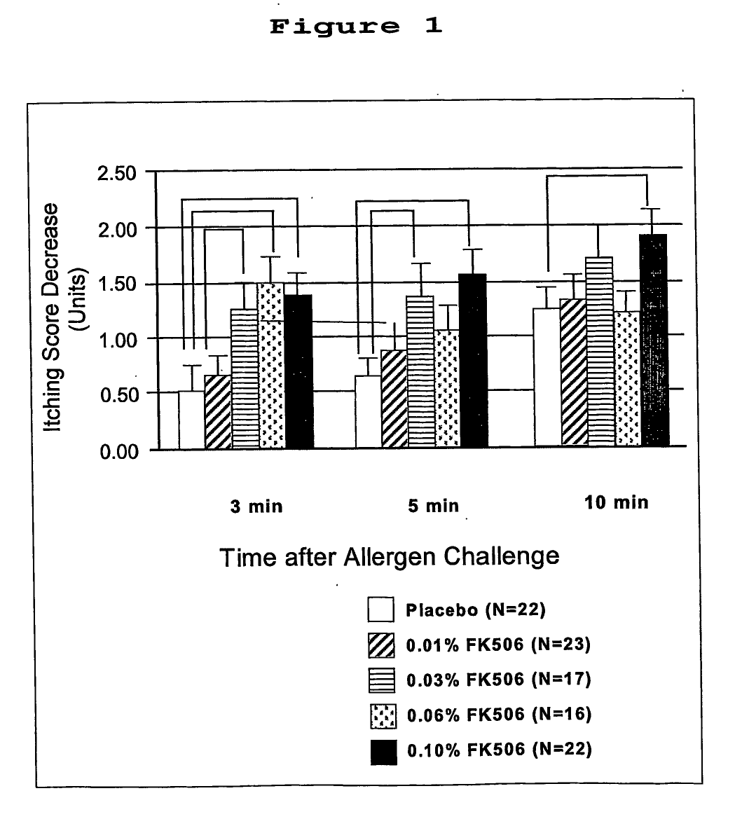

Human patients with a history of allergy were divided into 5 groups and treated in one eye with eye drop (placebo, 0.01% FK506, 0.03% FK506, 0.06% FK506 or 0.1% FK506), and the other eye with placebo. Each eye drop was administered 4 times per day for seven days and 16 hours after the final instillation, patients were administered allergen-containing eye drops at a concentration predetermined to cause a reaction in the patient. The one hundred patients having a baseline itching score of at lease 3, on a scale of 0 to 4, with 4 being most severe, were evaluated. Data of decrease from baseline itching score are presented in FIG. 1.

As seen in FIG. 1, there was a pronounced dose response, especially at 3 minutes post challenge, when all concentrations were statistically significant versus placebo.

This application is based on application No. 60/402,051 filed in United States of America, the content of which is incorporated hereinto by reference.

1. A method of treating a human patient suffering from ocular allergy, comprising administering to said patient an ophthalmic composition containing from about 0.01% to about 0.1% of macrolide compound.

2. A method according to claim 1 wherein said ocular allergy is allergic conjunctivitis.

3. A method according to claim 1 or 2 wherein said composition contains from about 0.03% to about 0.06% of said macrolide compound.

4. A method according to claim 3 wherein said macrolide compound composition contains about 0.03% of said macrolide compound.

5. A method according to claim 1 wherein said macrolide compound is FK506.

6. A method according to claim 1 wherein said ophthalmic composition is eye drop.

7. A method according to claim 6, wherein said eye drop further contains polyvinyl alcohol.

8. A method according to claim 7, wherein said eye drop contains about 0.03% of said macrolide compound.

9. A method according to claim 8, wherein said eye drop is administered from about one to about 4 times per day.

10. A method according to any of claims 1 to 9, wherein said macrolide compound is a compound having the following formula (I) or a pharmaceutically acceptable salt thereof: 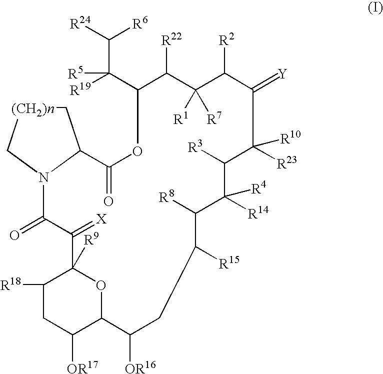
wherein adjacent pairs of R¹ and R², R³ and R⁴, and R⁵ and R⁶ each independently
a) consist of two adjacent hydrogen atoms, wherein R² is optionally alkyl, or
b) form another bond optionally between carbon atoms binding with the members of said pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkyl substituted by one or more hydroxy, alkenyl, alkenyl substituted by one or more hydroxy or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2.

11. A method according to claim 10, wherein said macrolide compound has the following structure:

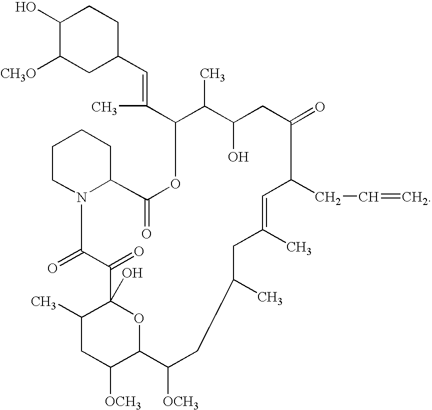

12. An ophthalmic composition for treatment of ocular allergy containing from about 0.01% to about 0.1% of macrolide compound.

13. An ophthalmic composition according to claim 12 wherein said ocular allergy is allergic conjunctivitis.

14. An ophthalmic composition according to claim 12 or 13 which contains from about 0.03% to about 0.06% of said macrolide compound.

15. An ophthalmic composition according to claim 14 which contains about 0.03% of said macrolide compound.

16. An ophthalmic composition according to claim 12 wherein said macrolide compound is FK506.

17. An ophthalmic composition according to claim 12 which is an eye drop.

18. An ophthalmic composition according to claim 17, wherein said eye drop further contains polyvinyl alcohol.

19. An ophthalmic composition according to claim 18, wherein said eye drop contains about 0.03% of said macrolide compound.

20. An ophthalmic composition according to claim 19, wherein said eye drop is administered from about one to about 4 times per day.

21. An ophthalmic composition according to any of claims 12 to 20, wherein said macrolide compound is a compound having the following formula (I) or a pharmaceutically acceptable salt thereof: 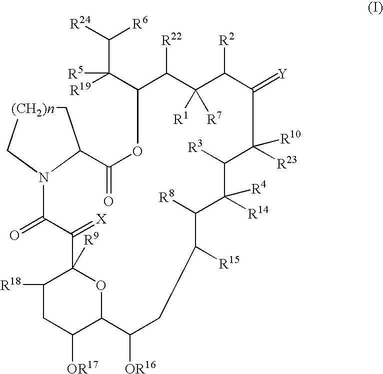
wherein adjacent pairs of R¹ and R², R³ and R⁴, and R¹ and R⁶ each independently
a) consist of two adjacent hydrogen atoms, wherein R² is optionally alkyl, or
b) form another bond optionally between carbon atoms binding with the members of said pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkyl substituted by one or more hydroxy, alkenyl, alkenyl substituted by one or more hydroxy or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2.

22. An ophthalmic composition according to claim 21, wherein said macrolide compound has the following structure:

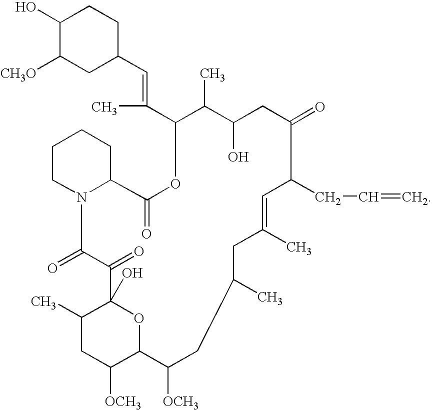

23. A use of macrolide compound for manufacturing an ophthalmic composition for treatment of ocular allergy, wherein said composition contains from about 0.01% to about 0.1% of said macrolide compound.

24. A use according to claim 23 wherein said ocular allergy is allergic conjunctivitis.

25. A use according to claim 23 or 24 wherein said composition contains from about 0.03% to about 0.06% of said macrolide compound.

26. A use according to claim 25 wherein said composition contains about 0.03% of said macrolide compound.

27. A use according to claim 23 wherein said macrolide compound is FK506.

28. A use according to claim 23 wherein said ophthalmic composition is an eye drop.

29. A use according to claim 28, wherein said eye drop further comprises polyvinyl alcohol.

30. A use according to claim 29, wherein said eye drop contains about 0.03% of said macrolide compound.

31. A use according to claim 30, wherein said eye drop is administered from about one to about 4 times per day.

32. A use according to any of claims 23 to 31, wherein said macrolide compound is a compound having the following formula (I) or a pharmaceutically acceptable salt thereof: 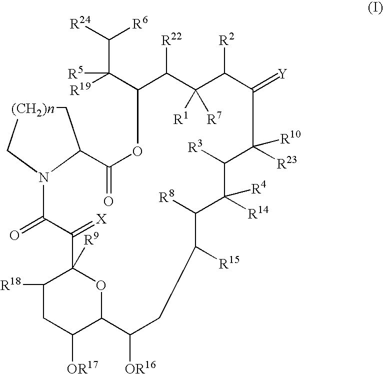
wherein adjacent pairs of R¹ and R², R³ and R⁴, and R⁵ and R⁶ each independently
a) consist of two adjacent hydrogen atoms, wherein R² is optionally alkyl, or
b) form another bond optionally between carbon atoms binding with the members of said pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkyl substituted by one or more hydroxy, alkenyl, alkenyl substituted by one or more hydroxy, or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2.

33. A use according to claim 32, wherein said macrolide compound has the following structure:

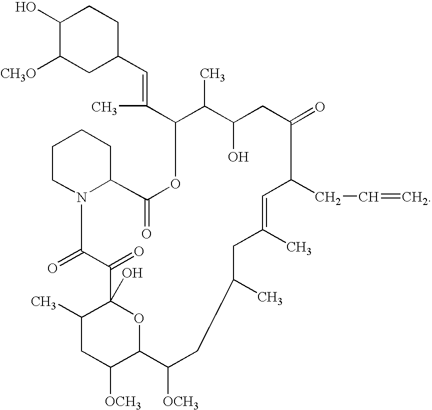

34. A commercial package comprising the ophthalmic composition of any of claims 12 to 22 and a written matter associated therewith, the written matter stating that the composition can or should be used for allergic conjunctivitis.
